(19) United States
(12) Patent Application Publication
(10) Pub. No.: US 2019/0295703 A1
(43) Pub. Date: Sep. 26, 2019

(54) PATIENT SELECTION BASED ON DRUG MECHANISM

(71) Applicants: Ritankar Das, Hayward, CA (US); Samson Mataraso, Hayward, CA (US); Jacob Calvert, Hayward, CA (US)

(72) Inventors: Ritankar Das, Hayward, CA (US); Samson Mataraso, Hayward, CA (US); Jacob Calvert, Hayward, CA (US)

(21) Appl. No.: 15/934,988

(22) Filed: Mar. 24, 2018

(51) Int. Cl. G16H 20/10; G06N 99/00; G16H 10/60

(52) U.S. Cl. CPC G16H 20/10; G16H 10/60; G06N 99/005

(57) ABSTRACT

Methods for identifying to which patients a drug should be administered, based on underlying drug mechanism of action, are provided. Machine learning techniques are used to determine that patient's underlying disease pathway includes drug mechanism of action target; the drug is then administered, based on this determination. Multiple types of data, including demographic, physiological, treatment, and clinical notes data, can be used to train a classification component. Multiple patient populations can be used as sources of patient data for training classification component. Data input requirements, dimensionality, and performance metrics may be optimized.

### FIELD OF THE INVENTION

The present disclosure generally relates to patient monitoring and diagnosis, and in particular to the design and implementation of clinical trials and the administration of drugs,

### BACKGROUND OF THE INVENTION

Clinicians have traditionally made decisions regarding which medication to prescribe for a patient's health condition or illness based on medication information that they have memorized or can quickly look up in a reference guide. The clinician may have learned about a given kind of medication, for example, a specific anti-inflammatory agent, from a medical journal, advertisement, educational lecture, or other means. Generally, drugs target specific physiologic pathways through their mechanism of action, and a disease which presents very similarly may be caused by different physiologic pathways. In certain heterogeneous disease populations (for example, sepsis, although there are many others), it would be valuable to know which drug to pick based on the underlying physiologic insult causing the disease. There currently exists limited ways to do this. One such way is a biomarker, but this requires a clinician to actively order a biomarker test, and many conditions do not reliable biomarkers that allow a clinician to learn about the underlying pathways causing the disease. Therefore, there exists a need for a method of deciding whether or not a drug will be effective based on underlying physiologic insult and whether or not the mechanism of action of the drug will treat that specific manifestation of the condition.

### SUMMARY OF THE INVENTION

The presently disclosed embodiments are directed to solving one or more of the problems presented in the prior art, described above, as well as providing additional features that will become readily apparent by reference to the following detailed description when taken in conjunction with the accompanying drawings.

In an embodiment, the disclosure provides a method of identifying a candidate patient for drug treatment, which includes acquiring patient data from a plurality of patients; comparing the acquired patient data to classified anonymized patient health record data; and identifying the candidate patient based on whether or not the drug's mechanism of action is efficacious in treating a specific manifestation of the candidate patient's condition as evidenced by the classified anonymized patient health record data.

In another embodiment, the disclosure provides a method of identifying a candidate patient for drug treatment, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

In another embodiment, the disclosure provides a method of identifying a candidate patient for drug treatment, wherein the anonymized patient health record data reflects the nature of the patient population served by the hospital or clinic in terms of patient demographics, rates of disease incidence, and/or treatment practices.

In another embodiment, the disclosure provides a method of identifying a candidate patient for drug treatment, wherein the anonymized patient health record data is sourced from a database of the plurality of patients, a database of one or more care centers and patient populations, or from a database of multiple care centers and patient populations.

In another embodiment, the disclosure provides a method of identifying a candidate patient for drug treatment, wherein the anonymized patient health record data is collected at a standard interval.

In another embodiment, the disclosure provides a method of identifying a candidate patient for drug treatment, wherein the anonymized patient health record data includes at least one patient labeled positive with respect to the gold standard which identifies that patient is progressing through a disease pathway for which the drug is expected to be effective.

In another embodiment, the disclosure provides a method of identifying a candidate patient for drug treatment, wherein the labeled patient data includes a positive label for a gold standard which represents a specific progression through the disease pathway.

In another embodiment, the disclosure provides a method of identifying a candidate patient for drug treatment, wherein the anonymized patient health record data continually improve as new data becomes available.

In another embodiment, the disclosure provides a method of identifying a candidate patient for drug treatment, wherein the classified anonymized patient health record data includes an operating point that balances measurements of specificity and sensitivity in order to effectively treat as many patients as possible.

In another embodiment, the disclosure provides a method of identifying a candidate patient for drug treatment, wherein the drug treatment includes administration of resatorvid, Zoptrex, eritoran, talactoferrin alfa, a 5-HT4 agonist, a TLR-4 inhibitor, a PCSK9 inhibitor, anacetrapib or thrombomodulin alfa or any salt, acid, base, hydrate, solvate, ester, isomer, polymorph, metabolite or prodrug thereof.

In another embodiment, the disclosure provides a method of using a machine learning algorithm for identifying a candidate patient for drug treatment, which includes acquiring anonymized patient health record data from a plurality of patients; comparing acquired patient data to the acquired anonymized patient health record data; and identifying the candidate patient based on whether or not the drug's mechanism of action is efficacious in treating a specific manifestation of the candidate patient's condition as evidenced by the anonymized patient health record data.

In another embodiment, the disclosure provides a method of using a machine learning algorithm for identifying a candidate patient for drug treatment, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

In another embodiment, the disclosure provides a method of using a machine learning algorithm for identifying a candidate patient for drug treatment, wherein the anonymized patient health record data reflects the nature of the patient population served by the hospital or clinic in terms of patient demographics, rates of disease incidence, and/or treatment practices.

In another embodiment, the disclosure provides a method of using a machine learning algorithm for identifying a candidate patient for drug treatment, wherein the anonymized patient health record data is sourced from a database of the plurality of patients, a database of one or more care centers and patient populations, or from a database of multiple care centers and patient populations.

In another embodiment, the disclosure provides a method of using a machine learning algorithm for identifying a candidate patient for drug treatment, wherein the anonymized patient health record data is collected at a standard interval.

In another embodiment, the disclosure provides a method of using a machine learning algorithm for identifying a candidate patient for drug treatment, wherein the anonymized patient health record data includes at least one gold standard patient data that identifies that patient is progressing through a disease pathway for which the drug is expected to be effective.

In another embodiment, the disclosure provides a method of using a machine learning algorithm for identifying a candidate patient for drug treatment, wherein the anonymized patient health record data includes at least one patient labeled positive with respect to the gold standard which identifies that patient is progressing through a disease pathway for which the drug is expected to be effective.

In another embodiment, the disclosure provides a method of using a machine learning algorithm for identifying a candidate patient for drug treatment, wherein the anonymized patient health record data continually improve as new data becomes available.

In another embodiment, the disclosure provides a method of using a machine learning algorithm for identifying a candidate patient for drug treatment, wherein the classified anonymized patient health record data includes an operating point that balances measurements of specificity and sensitivity in order to effectively treat as many patients as possible.

In another embodiment, the disclosure provides a method of using a machine learning algorithm for identifying a candidate patient for drug treatment, wherein the drug treatment includes administration of resatorvid, Zoptrex, eritoran, talactoferrin alfa, a 5-HT4 agonist, a TLR-4 inhibitor, a PCSK9 inhibitor, anacetrapib or thrombomodulin alfa or any salt, acid, base, hydrate, solvate, ester, isomer, polymorph, metabolite or prodrug thereof.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, size, or applicability of the disclosure. It should be noted that for clarity and ease of illustration these drawings are not necessarily made to scale.

FIG. 1 illustrates an embodiment of the different types of data that can be used that are relevant to the classification or prediction task;

FIG. 2 illustrates an embodiment of the possibilities for training on different populations of data;

FIG. 3 illustrates an embodiment of a gold standard determination of positive-class and negative-class labels;

FIG. 4 illustrates an embodiment of decision-making processes;

FIG. 5 illustrates an embodiment of example timelines of two progressions of the same disease;

FIG. 6 illustrates an embodiment of a timeline of disease progression; and

FIG. 7 illustrates an embodiment of an inclusion chart of a clinical trial run with an algorithmic companion diagnostic.

### DETAILED DESCRIPTION OF EXEMPLARY EMBODIMENTS

The following description is presented to enable a person of ordinary skill in the art to make and use embodiments described herein. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the disclosure. The word “exemplary” is used herein to mean “serving as an example illustration.” Any aspect or design described herein as “exemplary” is not necessarily to be construed as preferred or advantageous over other aspects or designs. Thus, the present disclosure is not intended to be limited to the examples described herein and shown but is to be accorded the scope consistent with the claims.

As used herein, reference to any biological drug includes any fragment, modification or variant of the biologic, including any pegylated form, glycosylated form, lipidated form, cyclized form or conjugated form of the biologic or such fragment, modification or variant or prodrug of any of the foregoing. As used herein, reference to any small molecule drug includes any salt, acid, base, hydrate, solvate, ester, isomer, or polymorph thereof or metabolite or prodrug of any of the foregoing.

It should be understood that the specific order or hierarchy of steps in the process disclosed herein is an example of exemplary approaches. Based upon design preferences, it is understood that the specific order or hierarchy of steps in the processes may be rearranged while remaining within the scope of the present disclosure. Any accompanying method claims present elements of the various steps in a sample order and are not meant to be limited to the specific order or hierarchy presented.

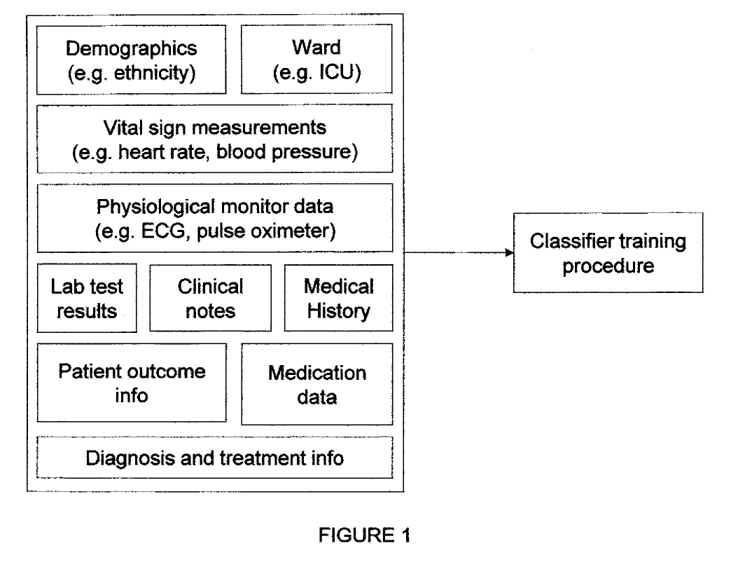

FIG. 1 illustrates an embodiment of the different types of data that can be used that are relevant to the classification or prediction task. The use of machine learning techniques necessitates the availability of data relevant to the classification or prediction task, on which to train a machine learning algorithm. In the context of the present disclosure, these data are typically anonymized patient health records, which can include amongst other information: (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and (x) patient medical history. Any or all of these types of data, along with other types of patient health information, can serve as inputs to the training procedure associated with a machine learning algorithm.

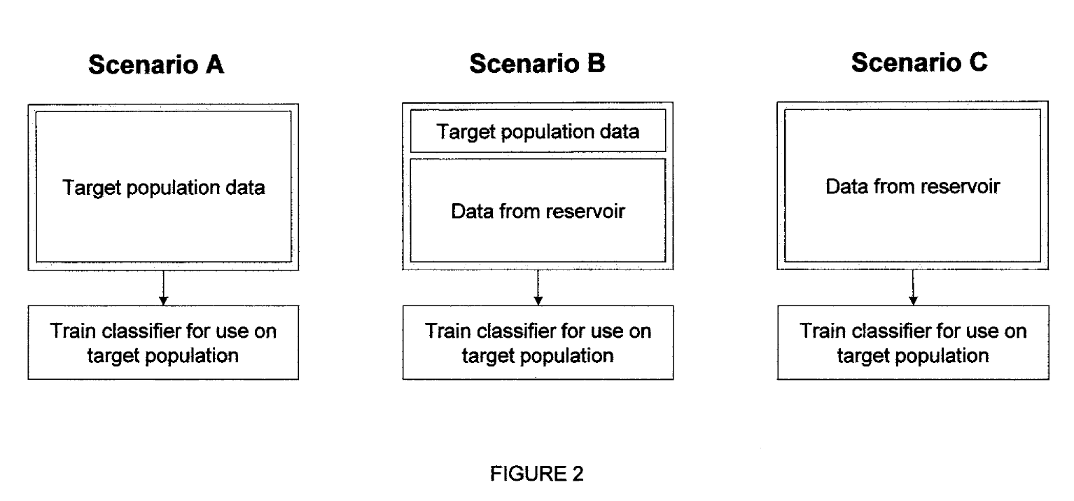

FIG. 2 illustrates an embodiment of the possibilities for training on different populations of data. Patient health record information can be collected and stored by the hospitals or clinics which deliver patient care. In this case, the health record data may reflect the nature of the patient population served by the hospital or clinic, in terms of patient demographics, rates of disease incidence, treatment practices, and other information potentially relevant to a prediction or classification task. Accordingly, when using a machine learning algorithm to develop a prediction or classification tool for use at a hospital or clinic, it may be useful to train with data from the relevant patient population or data from a similar population.

Patient health record data for training a machine learning algorithm can be sourced from a pre-existing or reservoir of data, including anonymized health records from one or more care centers and patient populations, typically with some variability in the types and amount of data that are available for patients in the data set.

Alternatively, health record data can be obtained from multiple care centers and patient populations. For example, the Medical Information Mart for Intensive Care III (MIMIC-III) is a publicly-accessible database of anonymized patient health record information, collected from Beth Israel Deaconess Medical Center (Boston, Mass.) between 2001 and 2012, which contains many of the aforementioned types of health data for tens of thousands of patients. A database like MIMIC-III would contrast with patient health record data available from the Veterans Health Administration, for example, which consists of many more health centers, spread across many states and cities. Accordingly, the types and resolution of health data available from such a data set would likely vary more.

When training a machine learning algorithm, it is typically ideal to train on a data set collected from the same population on which the resulting tool is intended to be applied. If there are sufficient training data from the target care center or population, the training procedure can proceed without modification as specified by the machine learning algorithm. If, however, there are not sufficient available data, the training procedure may be modified to rely on both a reservoir of health record data, as well as a small collection of clinic- or population-specific data; alternatively, the training procedure may rely entirely on a reservoir of data, A typical way to modify the training procedure in the former case is with the techniques of transfer learning, wherein the machine learning algorithm is first trained on a reservoir of data, before being trained further on the target dataset in such a way as to emphasize the examples it contains.

Ideally, all measurements relevant to a prediction or classification task are measured frequently, at a standard interval, e.g. one measurement every hour. However, patient health record data include types of data with varying frequencies of measurement and, as such, it is often convenient to standardize the frequencies with which new measurements are assessed by the prediction or classification tool resulting from the training procedure. For example, to produce a new patient classification every hour, the time series of measurements may be partitioned or “binned” into one-hour increments and relayed to the classifier accordingly.

As there will likely be bins during which no new measurement is available for a particular patient and type of data, it is standard to implement a data imputation scheme, whereby available data are used to fill-in missing data. The simplest such imputation method is a “carry-forward” rule, where the most recent measurement for a particular input, e.g. a heart rate measurement, is used in subsequent empty bins. There are other, more complicated methods for data imputation, including the filling of empty bins with the running average of the measurements of the relevant input, or inferring the missing value from a patient with a quantitatively similar trajectory of measurements.

It is also the case that sometimes multiple measurements of the same clinical variable are available within the same binning period. In this case, the frequency of measurements can be standardized by replacing the multiple measurements with the average of their values.

Supervised machine learning algorithms require labeled training data to identify the patterns in the data from which labels can be inferred. For example, to train a classifier for a sepsis from patient health record data, each patient must be assigned a positive or negative label, respectively indicating whether the patient did or did not have sepsis. Typically, before using unlabeled patient data with a supervised learning algorithm, the relevant label is assigned to the patient unambiguously in terms of the data that are available for that patient. This unambiguous way of assigning a label is called a gold standard.

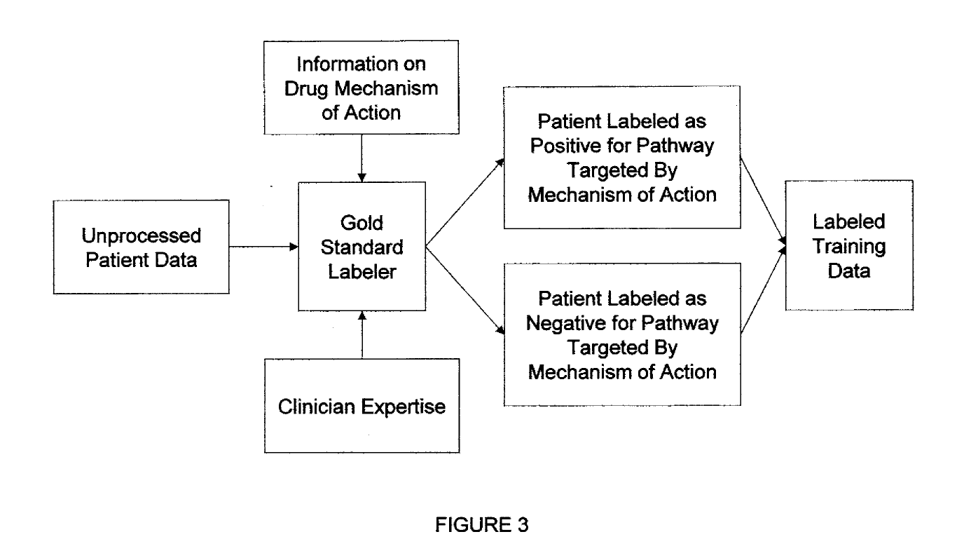

FIG. 3 illustrates an embodiment of a gold standard determination of positive-class and negative-class labels. In many cases, there is no universally agreed-upon gold standard for a label of medical relevance. Accordingly, a choice of gold standard may rely on a combination of standards of medical practice, correlative analyses, data availability constraints, and clinician expertise.

In the context of developing a tool for identifying candidates for drug administration, a relevant gold standard might produce a label indicating that a given patient is progressing through a disease pathway for which the drug is expected to be effective.

The drug resatorvid (TAK-242, ethyl (6R)-6-[(2-chloro-4-fluorophenyl)sulfamoyl]-cyclohexene-1-carboxylate), Takeda Pharmaceutical Company, Ltd.), for example, is a small molecule inhibitor of a receptor which initiates pro-inflammatory cytokine production. It is believed that resatorvid may improve sepsis outcomes by preventing cytokine storm. However, resatorvid is likely most effective when applied before the hyper-inflammatory stage of severe sepsis onset. As such, it is desirable to predict the onset of severe sepsis, and to include such a determination as a companion diagnostic to resatorvid. In this case, an appropriate choice of gold standard would label patients as being severely septic, so that the training procedure results in a prediction tool which can anticipate severe sepsis onset and therefore, determine to which patients resatorvid should be administered.

This training paradigm is different than training methodologies of other medical algorithms, which typically use a diagnosis of a disease as a gold standard, as opposed to using a specific progression through a disease pathway. These specific physiological phenomena will be identified through higher-level disease indicators.

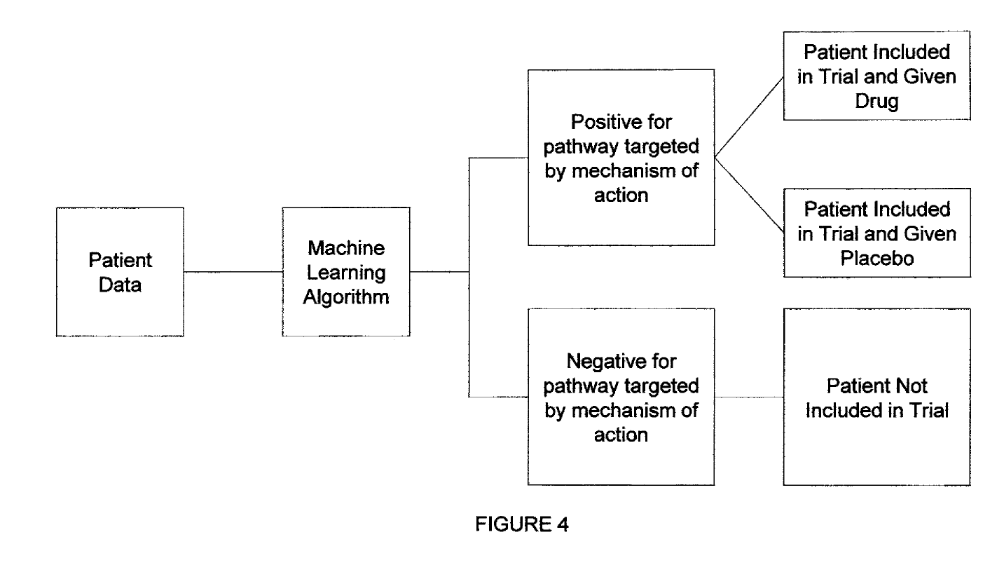

FIG. 4 illustrates an embodiment of a decision-making process, which includes (i) machine-learning-based tool determines that patient is in subset of disease progression pathways including mechanism of action target, (ii) patient is allocated uniformly-at-random to experimental or control groups of trial relevant clinicians are alerted to positive-class classification of patient, (iii) relevant clinicians administer drug to patient on the basis of positive-class classification.

The machine learning procedure identifies which features of the data set are most important for the classification or prediction task under consideration. Typically, in the context of the present disclosure, the features are the clinical variables, e.g. vital signs, lab test results, as well as their correlations, e.g. correlation between heart rate and blood pressure, and trends over time, e.g. differences in measurements taken at the beginning and end of a time window. However, it may also be the case that the features consist of all the data points of a patient's stay or, contrastingly, exclusively derivatives thereof.

If too many features are used in the learning procedure, training can be slow and may overfit the data. Overfitting leads to the appearance of good prediction performance, when tested with the data set on which it was trained, but results in poor generalization to other data sets, i.e. other patient populations. One way to prevent overfitting is by reducing the dimensionality, or number of features, included in the training procedure. Preliminary training and testing can identify those features that are most important to the prediction process. The less important features can then be removed from the training procedure. Another way to prevent overfitting is cross-validation where the data is randomly partitioned into a training set and a test data set as is known in the art.

The result of the training procedure is, in one form or another, a weighting of the features that can then be used to make predictions on new examples, subject to the features first being constructed from the new data. For classifying aspects of patient health, weighting the various features often leads to a numerical score that reflects the extent to which a given patient is believed to belong to a particular class. By placing a threshold on the score, e.g. patients whose scores are above 10 are determined to have sepsis; those with scores below 10 do not, they can be classified as either positive or negative for a given indication.

The machine learning algorithms can utilize information about a patient's current medical state, and also contextualize measurement information. The algorithm contextualizes by looking at deviation from a prior normal. Although there are medically accepted reference ranges for normal values of certain measurements, by analyzing prior measurements the algorithms determine what is normal for a specific patient.

Consider, for example, a drug designed to treat hypertension. Although a systolic blood pressure of 120 mm Hg is often considered normal, it represents hypertension in a patient who is regularly hypotensive, e.g. <100 mm Hg. As such, the algorithm can be trained to administer a hypertension drug at a systolic blood pressure of 120 mm Hg for a hypotensive patient, but not for other patients.

Training a machine learning algorithm must be done cognizantly, as the performance of the algorithm depends heavily on (i) the data used to train the algorithm; and (ii) the technique chosen. The data used to train the algorithm must labeled with a gold standard as described in the prior sections. Further, previously described various imputation and feature selection methods must be used to ensure the data is as well formed as possible. Well-formed data allow training processes to identify the best features for predicting patient response to various drugs.

Multiple learning techniques can be tested when developing the disclosed companion algorithms and the one that produces the best area under the receiver operating characteristic curve can be utilized. Simple techniques such as linear regression can be used, which attempts to find the best equation for a linear regression to fit to the data. Also tested are more complicated techniques, such as gradient boosted trees. Gradient boosted trees utilize multiple weak prediction models, in this case, decision trees. Decision trees are rule-based models which assign what is in effect a score based on an established set of rules. When combining many decision trees through gradient boosting, very robust predictions are often seen. Further, deep learning methods such as neural nets can be used to train the algorithm.

The disclosed algorithms can make suggestions about which patient should be receiving treatment based on whether or not a drug's mechanism of action would be efficacious in treating the specific manifestation of their condition and as patients are treated accordingly, the patient's health typically improves; however, it may not. In order to reduce false positives, which can cause alarm fatigue, there are inevitably patients that are not treated which should be. Likewise, in order to treat as many patients as possible, the disclosed algorithms sometime suggest treating patients for whom the drug is ineffective. This balance is achieved by selecting an appropriate operating point, which balances these two measurements, e.g. specificity and sensitivity.

As the disclosed algorithms run and patients are treated, more data is generated. Another training technique utilized is called online learning. Online learning allows algorithms to continually improve themselves as new data become available. In this context, the disclosed algorithms are able to learn from its own mistakes. If it suggests prescribing a drug to a patient that ultimately does not have the appropriate physiology to respond to the drug's mechanism of action, that patient will become part of the training data and improve the algorithm's future predictions. Online learning techniques, combined with transfer learning described above, can allow an algorithm to be customized to a particular patient if that patient is analyzed by the algorithm multiple times. Consider the cancer drug Zoptrex (zoptarelin doxorubicin, Aeterna Zentaris). In clinical trials, this drug was typically administered in multiple cycles. By utilizing information about a patient's response in a prior administration, the algorithm can more accurately predict how a patient will respond to the drug in future administrations. Online learning can allow an algorithm to become more robust in determining if a drug will be effective as patients as given the drug, which is especially powerful in drugs that are typically administered in cycles and may not have clear effects immediately, such as Zoptrex.

There are many settings available for selection of an operating point, which can be indicated as points along an ROC curve. The selection of an operating point is at the discretion of the user. It is a trade-off of sensitivity and specificity; this connects with the number of alerts a user would like to have over a period of time.

Although these algorithms are developed to predict a binary outcome—whether or not a patient has a certain physiologic condition upon which a certain drug can act—they are trained to output some score along a scale. The binary outcome is achieved by choosing an operating point. However, by selecting multiple operating points, the disclosed algorithms can be generalized to scenarios in which there are multiple options. Depending on the range that a patient's score falls within, a specific course of clinical treatment can be suggested.

Consider for example drug dosing. If a patient is likely to have a certain physiological phenomenon that aligns with a drug's mechanism of action, the disclosed algorithms can be designed to administer the whole dose of the drug. However, if the disclosed algorithm's score for a particular patient falls within some grey area, i.e. close to the operating point in a binary decision, then administering a lesser dose of the drug, measuring patient outcome, and monitoring the patient for improvement can be used, Whether or not the patient improves will allow the algorithm to better determine whether or not the patient had the specific physiologic phenomenon and suggest appropriate treatment. Further, this label can be used in conjunction with the online learning scheme described above.

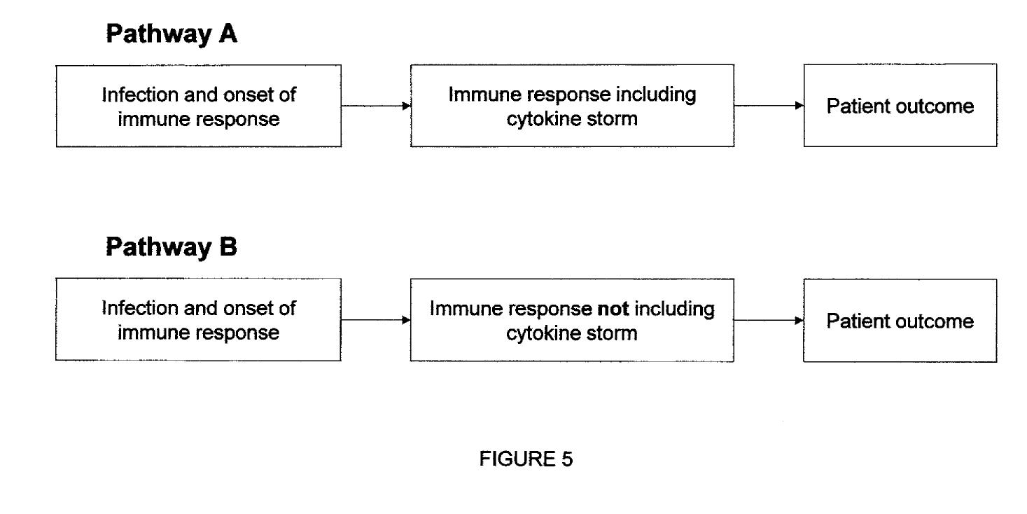

FIG. 5 illustrates an embodiment of example timelines of two progressions of the same disease. Specifically, look at the drug resatorvid. In a pivotal phase 3 clinical trial, patients received one of two doses of the drug. A stronger effect was seen in the higher dose arm; however, the drug also has side effects. By utilizing the ability of the algorithm to tackle non-binary problems, it can make suggestions as to whether or not a patient should receive a low dose or high dose based on their cytokine activity.

In clinical settings, the disclosed algorithms can be implemented directly within an EHR. This direct implementation allows for algorithms to process data in real time from patients as it is entered into their medical records. Further, alerts will be able to be displayed directly to clinicians in a patient's chart. However, external alerts, such as phone calls, are also possible through integration with automated calling APIs. When the disclosed algorithms detect that a patient has a certain condition that has manifested with a physiology that is conducive to being treated with a drug, it will alert a clinician that (i) they have a certain condition and (ii) a certain drug should be prescribed.

The disclosed algorithms can be used to improve clinical trials. Often times, drugs are effective, but their results in clinical trials are underpowered. By selecting patient responders that align with the mechanism of action of a drug, this efficacy signal can be strengthened, resulting in a successful trial. In a clinical trial, the disclosed algorithms can be used as inclusion criteria. Currently, patients are enrolled in a trial if they meet the rule-based criteria, which often attempts to, in a rudimentary fashion, detect the physiological mechanism the drug treats. However, algorithmic inclusion criteria provide a more robust way of confirming that a certain patient's condition is likely to be treated by a drug. Upon a positive result from the disclosed algorithms, the algorithms can alert a patient's clinicians that the patient (i) has a condition which makes them eligible for enrollment in a clinical trial; and (ii) their condition is likely to be treated by the drug being tested in their trial.

Consider the TLR-4 inhibitor class of drugs, for example eritoran (([(2R,3R,4R,5S,6R)-4-Decoxy-5-hydroxy-6-[[(2R,3R,4R,5S,6R)-4-[(3R)-3-methoxydecoxy]-6-(methoxymethyl)-3-[[(Z)-octadec-11-enoyl]-amino]-5-phosphonatooxyoxan-2-yl]oxymethyl]-3-(3-oxotetradecanoyl-amino)oxan-2-yl] phosphoric acid), Eisai Inc.) or resatorvid. These drugs interact with lipopolysaccharides, which are present in gram-negative bacteria. As such, there is a value in detecting patients with infections caused by gram-negative bacteria. The disclosed algorithm can be trained to detect these patients, and TLR-4 inhibitors can be selectively administered to only these patients, thus improving efficacy signals. Thus, the disclosed algorithms can determine if patients with infection have an infection due to gram positive or gram negative bacteria, which can be used to better prescribe TLR-4 inhibitors.

Consider for example the drug talactoferrin alfa (a type of recombinant protein and a type of immunomodulatory protein, also called talactoferrin and TLF, Agennix Inc.). Clinical trials of talactoferrin alfa for the treatment of sepsis have found that it causes higher mortality rates with septic shock, but a slightly lower mortality rate in patients without shock. The disclosed algorithm can be trained to detect patients that have an underlying physiology likely to lead to sepsis without shock. These patients may respond better to talactoferrin alfa. By targeting these patients, the drug's risk-benefit profile can be improved, potentially improving the results of a clinical trial. Thus, the disclosed algorithms can predict patients who are likely to develop severe sepsis (as opposed to just sepsis) to administer resatorvid or talactoferrin alfa to patients.

Additionally, consider the 5-HT4 agonist class of drugs for the treatment of enteral feeding intolerance (EFI). Often times, a cause of EFI is issues with gastrointestinal (GI) motility. The 5-HT4 class of drugs aims to restore GI motility. By training the disclosed algorithm to detect patients whose EFI resulted from loss of GI motility, a 5-HT4 agonist can be given only to patients for whom the drug would be effective. Further, the 5-HT4 agonist can be given to any patient who has GI motility issues, regardless of it develops into EFI. Thus, the disclosed algorithms can determine if patients have issues with gastrointestinal motility to prescribe 5-HT4 agonists.

Consider the drug anacetrapib ((4S,5R)-5-[3,5-bis(trifluoromethyl)phenyl]-3-({2-[4-fluoro-2-methoxy-5-(propan-2-yl)phenyl]-5-(trifluoromethyl)phenyl}methyl)-4-methyl-1,3-oxa-zolidin-2-one, Merck) to treat patients with atherosclerotic vascular disease. Anacetrapib is a cholesteryl ester transfer protein (CETP) inhibitor. PCSK9 inhibitors are another class of drug which can be used to treat atherosclerotic vascular disease (among other conditions). However, some patients may respond different to CETP inhibitors than to PCSK9 inhibitors. Thus, the disclosed algorithms can determine if patients are more likely to respond to a CETP inhibitor or a PCSK9 inhibitor to determine which class of drugs is best to use to treat atherosclerotic vascular disease

The disclosed algorithm can be trained to detect physiological insults leading to high cholesterol levels and predict which class of drugs would be more effective for the treatment of the patient. As such, drugs from these classes can be prescribed more specifically, resulting in improved patient outcomes. Thus, the disclosed algorithms can determine the severity of an underlying physiologic insult and be used to more specifically dose patients.

Consider the drug thrombomodulin alfa, a recombinant and soluble thrombomodulin (ART-123, Asahi Kasei Pharma Corp.), a drug that has been hypothesized to have the ability to treat patients with disseminated intravascular coagulation (DIC) in response to infection. DIC is difficult to characterize; machine learning lends itself to detecting conditions which are difficult to characterize, as its statistical foundations are able to detect complicated patterns or subtle signals. As such, the disclosed algorithm could be used to detect which patients have DIC, thus allowing thrombomodulin alfa to be administered more selectively to the patients it is intended to treat.

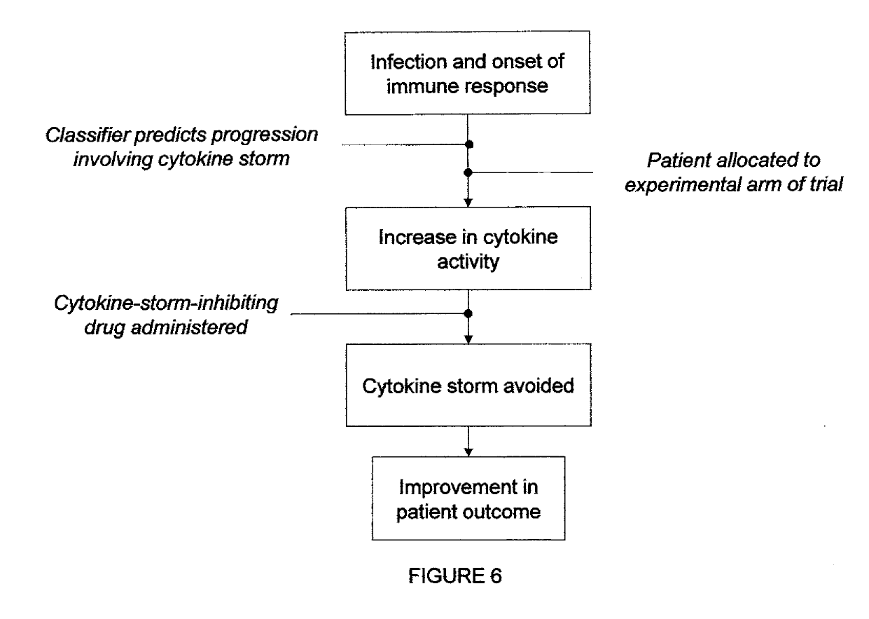

FIG. 6 illustrates an embodiment of a timeline of disease progression. There are applications to improve trials as a whole. There is a process of informed consent in clinical trials that is often lengthy. This time delay required to educate a patient and have them consent can be fatal. For example, sepsis is a disease which progresses over the course of a few hours or a few days. Delaying treatment substantially increases mortality. The disclosed algorithms can detect an increasing trend in a patient's score and predict that they will develop a condition in the near future. The disclosed algorithms can alert trial managers to speak with the patient and pre-consent, meaning that in the likely event they do develop a certain condition, such as sepsis, precious time will not be wasted by having them fully consent.

Increasingly, clinical trials utilize adaptive trial designs. Adaptive trial designs begin by looking at a drug's effect in a smaller subgroup of patients to determine likely effect size. Then, as needed, the trial is expanded to more patients to show statistical significance. A smaller initial trial size allows trial coordinators to terminate the trial if initial results show either futility or efficacy. The algorithms described can be used to improve adaptive trial design. By constantly performing interim analyses it can continuously update needed trial size to show efficacy, preventing unnecessary costs due to over enrollment in the trial. Further, the ability to select responders results in a stronger efficacy signal, meaning less patients need be enrolled to show statistical significance. As such, trial size and length can decrease, resulting in substantial cost reduction.

Fundamentally, these algorithms are a method of selecting patient responders. They serve as companion diagnostics in the same way that a more traditional biomarker or genetic test would. However, they improve upon these methods in multiple ways. Companion diagnostics such as traditional biomarkers and genetic tests require an active order from a clinician, which can not only take time but also may be overlooked. An algorithmic companion diagnostic is passive, and always monitoring patients, which means as soon as they develop some physiologic condition that can be treated by a drug, they are diagnosed thusly.

Further machine learning is a powerful tool for prediction. By analyzing patient trajectories, the disclosed algorithms have the ability to predict the onset of disease, in addition to detecting it. Therefore, companion diagnostics may be able to diagnose conditions earlier.

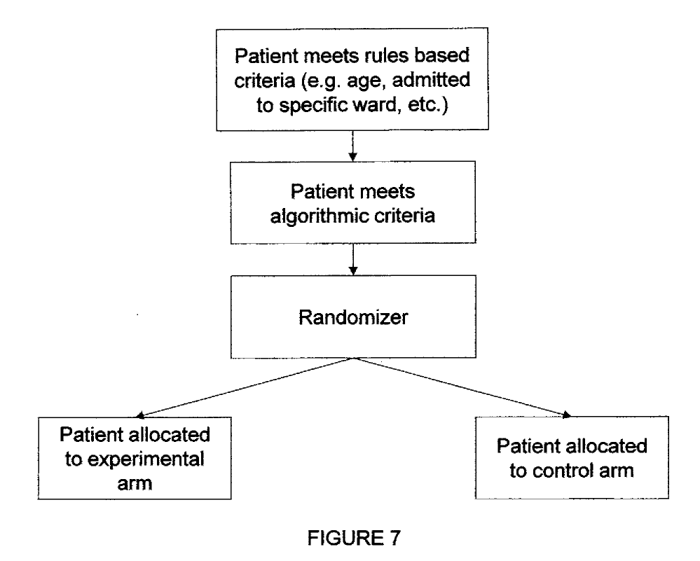

FIG. 7 illustrates an embodiment of an inclusion chart of a clinical trial run with an algorithmic companion diagnostic. Algorithms can also be developed with data. These data are already collected at thousands of hospitals across the country. There are no expensive R&D procedures necessary as are often the case with biomarkers and genetic tests. Further, they can be validated on partitioned data—no in vitro tests are required. Lastly, the ability to analyze features which represent more complicated bodily functions, e.g. by being composed of multiple vital signs and lab tests, leads to these algorithms typically having more discriminatory power. Because the algorithm is an inclusion criterion for the trial, it would become part of the drug's indication upon regulatory approval.

Because the specific physiologic phenomena and thus the gold standards used for labeling data, are often not explicit outcomes present in the EHR, there is a substantial barrier to acquiring labeled data that can be used to train these algorithms. This issue can be solved in a number of ways. First, clinicians can hand-label charts for the presence of the pathway targeted by the mechanism of action of the drug. Manual labeling of charts can be incredibly valuable for creating a training dataset which is accurately labeled. Using these data as a base can allow an algorithm to be trained enough to produce good results, which can be further improved by online learning.

Additionally, drugs often fall into a specific class, for example resatorvid and eritoran are both TLR-4 inhibitors. Because drugs within the same class typically have identical or similar mechanisms of action, transfer learning techniques can be applied utilizing data from patients who have received drugs of the same class.

After implementing a classifier resulting from the training procedure, it may be desirable to update the classifier to reflect different priorities of use or to reflect new patient data that have become available for training. Retraining can be completed in batches, that is, by performing the training procedure on an updated training set and choosing an operating point to reflect the use priorities, i.e. picking the sensitivity and specificity of alerts clinicians can expect to receive, which determines the number of alerts clinicians can expect to receive, in the same way as was originally done. Retraining can also be completed continuously as new data become available using an online machine learning technique.

The ability to identify a specific physiologic pathway has broad implications. As described above, an algorithm with discriminatory ability to detect a mechanism of action can increase efficacy signal in a drug for a specific indication. However, the algorithm can also be applied to new indications. Often, drugs work for a specific disease indication because a physiologic pathway is commonly associated with the disease. Utilizing the power of being able to detect a certain physiologic pathway means that a drug's indication can be expanded to other diseases, including those where such a pathway is less common. While traditionally a drug would not be able to show efficacy in a heterogenous disease that can manifest through different physiologic insults, selecting patient responders based on those exhibiting signs of a certain insult can allow efficacy to be reached. Further, it may be possible to have a label based solely upon a disease mechanism, unrelated to any known conditions. This would allow a drug to be used to treat rare or previously undiagnosed conditions, so long as a patient is likely to be helped by the drug.

While the inventive features have been particularly shown and described with reference to preferred embodiments thereof, it will be understood by those in the art that the foregoing and other changes may be made therein without departing from the sprit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

What is claimed is:

1. A method of identifying a candidate patient for drug treatment, comprising:
acquiring patient data from a plurality of patients;
comparing the acquired patient data to classified anonymized patient health record data; and
identifying the candidate patient based on whether or not the drug's mechanism of action is efficacious in treating a specific manifestation of the candidate patient's condition as evidenced by the classified anonymized patient health record data.

2. The method of claim 1, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

3. The method of claim 1, wherein the anonymized patient health record data reflects the nature of the patient population served by the hospital or clinic in terms of patient demographics, rates of disease incidence, and/or treatment practices.

4. The method of claim 1, wherein the anonymized patient health record data is sourced from a database of the plurality of patients, a database of one or more care centers and patient populations, or from a database of multiple care centers and patient populations.

5. The method of claim 1, wherein the anonymized patient health record data is collected at a standard interval.

6. The method of claim 1, wherein the anonymized patient health record data includes at least one patient labeled positive with respect to the gold standard which identifies that patient is progressing through a disease pathway for which the drug is expected to be effective.

7. The method of claim 6, wherein the labeled patient data includes a positive label for a gold standard which represents a specific progression through the disease pathway.

8. The method of claim 1, wherein the anonymized patient health record data continually improve as new data becomes available.

9. The method of claim 1, wherein the classified anonymized patient health record data includes an operating point that balances measurements of specificity and sensitivity in order to effectively treat as many patients as possible.

10. The method of claim 1, wherein the drug treatment includes administration of resatorvid, Zoptrex, eritoran, talactoferrin alfa, a 5-HT4 agonist, a TLR-4 inhibitor, a PCSK9 inhibitor, anacetrapib or thrombomodulin alfa.

11. A method of using a machine learning algorithm for identifying a candidate patient for drug treatment, comprising:
acquiring anonymized patient health record data from a plurality of patients;
comparing acquired patient data to the acquired anonymized patient health record data; and
identifying the candidate patient based on whether or not the drug's mechanism of action is efficacious in treating a specific manifestation of the candidate patient's condition as evidenced by the anonymized patient health record data.

12. The method of claim 11, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

13. The method of claim 11, wherein the anonymized patient health record data reflects the nature of the patient population served by the hospital or clinic in terms of patient demographics, rates of disease incidence, and/or treatment practices.

14. The method of claim 11, wherein the anonymized patient health record data is sourced from a database of the plurality of patients, a database of one or more care centers and patient populations, or from a database of multiple care centers and patient populations.

15. The method of claim 11, wherein the anonymized patient health record data is collected at a standard interval.

16. The method of claim 11, wherein the anonymized patient health record data includes at least one gold standard patient data that identifies that patient is progressing through a disease pathway for which the drug is expected to be effective.

17. The method of claim 16, wherein the at least one gold standard patient data includes a specific progression through the disease pathway.

18. The method of claim 11, wherein the anonymized patient health record data continually improve as new data becomes available.

19. The method of claim 11, wherein the classified anonymized patient health record data includes an operating point that balances measurements of specificity and sensitivity in order to effectively treat as many patients as possible.

20. The method of claim 1, wherein the drug treatment includes administration of resatorvid, Zoptrex, eritoran, talactoferrin alfa, a 5-HT4 agonist, a TLR-4 inhibitor, a PCSK9 inhibitor, anacetrapib or thrombomodulin alfa.
